(19) United States
(12) Patent Application Publication
(10) Pub. No.: US 2015/0148809 A1
(43) Pub. Date: May 28, 2015

(54) DYNAMIC CORPECTOMY CALIPERS AND METHODS OF USE THEREOF

(71) Applicant: Institute for Musculoskeletal Science and Education, Ltd., Radnor, PA (US)

(72) Inventor: Gregory Bailey, State College, PA (US)

(21) Appl. No.: 14/554,447

(22) Filed: Nov. 26, 2014

Related U.S. Application Data
(60) Provisional application No. 61/909,804, filed on Nov. 27, 2013.

(51) Int. Cl. A61B 5/107

(52) U.S. Cl. CPC A61B 5/1076
USPC 606/102

(57) ABSTRACT

Corpectomy calipers are provided that are capable of measuring both the distance and the angle between two bodies, in particular between two vertebral bodies in an individual. The calipers provided are capable of providing reliable measurements without the need for repeated measurements. The calipers described herein offer a simple and inexpensive solution. Methods of using the calipers are provided, for example, in determining the size and spacing of a void in a corpectomy or intervertebral fusion procedure.

### CROSS-REFERENCE TO RELATED APPLICATIONS

This application claims priority to U.S. Provisional Application No. 61/909,804, filed Nov. 27, 2013, the disclosure of which is incorporated herein by reference in its entirety.

### FIELD OF THE INVENTION

The present disclosure is generally in the field of calipers, in particular calipers for measuring the intervertebral space and methods of use thereof, for instance in a corpectomy procedure.

### BACKGROUND OF THE INVENTION

The spinal column in humans has a series of thirty-three stacked vertebrae divided into five regions. The spinal discs, the vertebral bodies, or both can be displaced or damaged due to trauma, disease, degenerative defects, or wear. More than 60% of patients beyond age 40 display some level of disc degeneration on an MRI. This is most prevalent in the lumbar spine.

A discectomy is a surgical procedure to remove herniated disc material that presses on a nerve root or on the spinal cord. Discectomy involves removing the central portion (nucleus pulposus) of an intervertebral disc that is stressing the spinal cord or radiating nerves, causing pain. A laminectomy or corpectomy is often performed in conjunction with a discectomy, for example to permit access to the intervertebral disc or to further to decompress the spinal cord and nerves. In a laminectomy, a small piece of bone (the lamina) is removed from the adjacent vertebra, allowing the surgeon to better see and access the area of disc herniation.

A corpectomy also involves removing part of the vertebral body. The approach for anterior cervical corpectomy is similar to an anterior cervical discectomy, although a larger and more vertical incision in the neck is often made to allow more extensive exposure. The surgeon typically performs a discectomy at either end of the vertebral body that will be removed. More than one vertebral body may be removed (a multi-level corpectomy).

After a corpectomy has been performed, the surgeon needs to mechanically reconstruct the void created and provide long-term stability of the spine with a spine fusion. A strut graft is a piece of bone inserted into the trough created by the corpectomy and that supports the anterior vertebral column. The graft may be an allograft or an autograft, and is usually then followed by anterior rod or plate instrumentation to help hold the construct together. Intervertebral cages made of titanium or other synthetic materials may be employed as an alternative to grafts.

Technically, a corpectomy is a difficult spine surgery to perform. The risk that spine surgeons worry about the most is compromise of the spinal cord that can lead to complete or partial quadriplegia. Corpectomy surgeries are often undertaken in circumstances of significant spinal cord problems, which place the cord at greater risk for problems during surgery, independent of the skill and finesse with which the procedure is performed. The risks and possible complications of this surgery for cervical spinal stenosis include nerve root damage, damage to the spinal cord, bleeding, infection, graft dislodgment, damage to the trachea/esophagus, and/or continued pain.

In the U.S., it has been estimated that the Medicare system spends over $300 million annually on lumbar discectomies (Schoenfeld et al., Int. J. of General Med., 2010, 3:209-214). A study of 1560 patients undergoing cervical corpectomy revealed an overall in-hospital mortality rate of 1.6%, a complication rate of 18.4%, and a mean length of stay of 6 days. Duration of surgery was observed as a significant predictor of postoperative complications. Furthermore, patients who were returned to the operating room had significantly higher mortality rates (7.0% versus 1.2%) and accounted for about 40% of the total number of complications. See Boakye et al., Neurosurgery, 2008, 4:295-301. An improper fit of the corpectomy cage in the void can be a major source of post-operative complications that require additional operations to repair.

Whether using anterior devices, such as rods or plates, or using intervertebral cages, the reconstructed void should be sized and shaped with precision to maintain mobility and function of the spinal column while providing the necessary structural support. In determining the dimensions for an intervertebral prosthesis, the overall height of the intervertebral prosthesis and an angular orientation of the surfaces are decisive. Typically, measurements of the intervertebral prosthesis are taken preoperatively using X ray or CAT imagery, and during the operation are verified after removal of the disk by test use of implants. Implant measurements are tested by inserting a test implant between the vertebral bodies mechanically spread apart from each other; the vertebral bodies are released; and then, using an X ray image, the fit of the implant is checked. This process requires spreading the vertebral bodies multiple times to remove and re-insert the test prostheses for insertion and checking of the intervertebral prostheses. This takes up a large part of the time for surgery; and necessitates repeated and excessive mechanical loads on the patient's spinal column. Devices that have been proposed to date to improve corpectomy procedures include complicated solutions that can be difficult and expensive to manufacture (if not to use). U.S. Pat. No. 8,282,650 to Weber and U. S. Patent Application Publication No. 2006/0074431 by O'Neil et al. describe such solutions. Procedures and devices for measuring the size and shape of the void during the operation that do not require multiple measurements or the placement of multiple test devices are needed.

It is therefore an object of the invention to provide tools and methods that simplify vertebral surgery, in particular vertebral corpectomy surgeries.

It is further an object of the invention to provide tools and methods that reduce operative time for vertebral surgical procedures, in particular vertebral corpectomy procedures.

It is also an object of the invention to tools and methods that aid surgeons in selecting intervertebral devices, such as intervertebral cages, that best fit a void or defect space.

Additional objects of the invention include providing tools and methods that improve patient success and reduce the rate of complications requiring post-operative return to the operating room.

### SUMMARY OF THE INVENTION

Dynamic corpectomy calipers provided herein are capable of measuring both the distance and the angle between two bodies, in particular between two vertebral bodies. The calipers provide simple alternatives to more complicated measurement devices known in the art, while providing the ability to measure in a single measurement both the distance and angulation between two vertebral bodies. The devices include a caliper portion for measuring the distance between a pair of contacting surfaces. In preferred embodiments the caliper portion is a traditional scissor-like caliper. The scissor-like caliper devices are reliable, easy to use, and simple to manufacture.

In preferred embodiments, the contacting surfaces extend from the caliper portion by a pair of arms. The arms are maintained in a substantially parallel orientation such that the caliper portion can readily measure the distance between the contacting surfaces. Typically one or both of the contacting surfaces are on movable contacting members capable of adjusting their angle to fit the measured space. In some embodiments, having a single movable contacting member and a single fixed contacting member simplifies measurement of the angle.

Methods of using the corpectomy calipers are provided for measuring the heights and angles of intervertebral spaces and voids. The methods are useful, for example, for choosing an intervertebral cage or a corpectomy cage that accommodates the space or void. The devices make possible the determination of both the distance and the angulation in a single measurement, thereby alleviating the time incurred by multiple measurements required in other procedures.

### BRIEF DESCRIPTION OF THE DRAWINGS

FIGS. 1A-F depict different views of an exemplary dynamic corpectomy caliper having a scissor-like caliper portion and a contacting portion. FIG. 1A is a perspective view of the dynamic corpectomy caliper in the closed position. FIG. 1B is a perspective view of the caliper in the open or extended position. FIG. 1C is an exploded view of the caliper depicting the components of the caliper portion and the contacting portion. FIG. 1D is a perspective view from the proximal end of the caliper depicting the mechanical gauges for reading both the distance and the angulation. FIG. 1E is a detailed sectional view of the distal end of the caliper depicting the contacting surfaces and mechanical gauge for measuring the angle. The caliper is in the closed position. FIG. 1F is a detailed sectional view of the distal end of the caliper depicting the contacting surfaces and mechanical gauge for measuring the angulation. The caliper is in the open or extended position.

### DETAILED DESCRIPTION OF THE INVENTION

### I. Dynamic Calipers

Dynamic corpectomy calipers that are relatively inexpensive to manufacture and simple to use are provided herein. The calipers are capable of simultaneously measuring both the height and the angle of an intervertebral void without the need for multiple measurements. The devices have a caliper portion that determines and measures the distance between a pair of contacting surfaces located in a contacting portion of the device. The contacting portion generally includes a pair of contacting surfaces and a means for measuring the angle between the contacting surfaces. While devices can be designed with various types of gauges known in the art, in a preferred set of embodiments the devices do not contain any digital gauges.

a. Caliper Portion

The term, “caliper portion”, as generally used herein, refers to a portion of the device that is a traditional caliper and is used to measure a distance that correlates to the distance between the contacting surfaces on the contacting end of the device. Calipers are generally known in the art and can either be a simple “scissor-like” device or a vernier caliper. The scissor-like calipers include a pair of arms pivotally engaged such that the angle transcribed by the arms correlates with the distance between the ends of the arms. The term “scissor-like caliper”, as generally used herein, refers to any calipers that contain a pair of arms pivotally connected at a point wherein the distance between the arms at some position away from the point is determined by the angle between the arms. The arms of a “scissor-like” caliper may include a handle portion and thereby resemble a pair of scissors in design, although this need not be the case.

A vernier caliper is more complicated that a scissor-like caliper and includes a pair of jaws, typically one of which is fixed and one of which is movable. The fixed jaw is attached to an elongated scale slidably engaged with the moveable jaw, whereby the position of the movable jaw relative to the fixed jaw can be determined by the position on the scale. In preferred embodiments, the caliper portion is a scissor-like caliper and is not a vernier caliper.

Scissor like calipers contain two arms pivotally engaged. The arms can be pivotally engaged, for example, by a pin, screw, or other suitable component as are known in the art. In some embodiments the arms are engaged via a hinge-like device. The caliper portion may include one or more handles for operating and maneuvering the device. The handles can be formed by a part of the arms, or may be separate elements attached to the caliper portion.

Calipers are useful for accurately measuring the distance between two points or surfaces. In some embodiments the caliper portion includes a digital gauge for displaying the distance between the contacting surface and/or relaying the distance between the contacting surfaces to another device capable of receiving digital information. In preferred embodiments, the caliper portion does not contain any digital gauges. The caliper portion can contain a mechanical gauge whereby the position of one element with respect to the other is indicative of the distance between the contacting surfaces. For example, a scissor-like caliper can contain an elongated member having a graduated scale fixed to one arm and an indicator fixed to the other arm. The position of the indicator with respect to the graduated scale can thereby be calibrated to the distance between the contacting surfaces. In preferred embodiments the gauges are mechanical gauges containing few moving parts.

The caliper portion is capable of measuring the distance between the contacting surfaces. The distances are typically in the range of 1-200 mm, preferably 5-150 mm, more preferably 5-100 mm. The caliper portion is capable of measuring the distance between the contacting surfaces with a precision of less than 1 mm, preferably less than 0.1 mm, more preferably less than 0.01 mm.

b. Contacting Portion

The contacting portion is operably engaged with the caliper portion of the device and includes at least a pair of contacting surfaces. One or both of the contacting surfaces may be on a contacting member such as a plate, disc, block, or the like having at least one surface substantially planar for contacting the region to be measured. One or both of the contacting members is hingedly engaged in such a manner that it can adapt to the angulation of the space to be measured. In a preferred embodiment one of the contacting members is fixed and only one contacting member is movable. This simplifies the measurement of the angle between the contacting surfaces. The angulation of the movable contacting member can be indicative of the angle between the first contacting surface and the second contacting surface.

The angle between the contacting surfaces can be measured by mechanical or digital devices. In some embodiments the contacting portion includes a digital gauge for displaying the angle between the contacting surfaces and/or relaying the angle between the contacting surfaces to another device capable of receiving digital information. In preferred embodiments, the contacting portion does not contain any digital gauges. The contacting portion can contain a mechanical gauge whereby the position of one element with respect to the other is indicative of the angle between the contacting surfaces. For example, the contacting portion can contain an indicator needle that is slidably engaged with an indicator panel having a graduated scale calibrated such that the position of the indictor needle relative to the graduated scale is indicative of the angle of the contacting surface.

The contacting portion includes a means for measuring the angle between the contacting surfaces. The angles are typically in the range of −45° to +45°, preferably −30° to +30°, more preferably −15° to +15. The caliper portion is capable of measuring the angulation between the contacting surfaces with a precision of less than 2°, preferably less than 1°, more preferably less than 0.1°.

The devices described herein can be manufactured by any method known to those skilled in the art. The individual components of the devices can be precision machined from materials safe for use in surgical instruments. The materials are generally known in the art. Metallic materials can be preferred for their resistance to wear. Components can be made from metallic materials by techniques such as casting and machining. In some embodiments the components are manufactured from stainless steel. Stainless steels which are suitable for use in surgical instruments are known.

The handle portion may include one or more polymeric or elastomeric materials that provide improved grip for the surgeon. Polymers which can be used in the handle include polyolefins such as polyethylene and polypropylene, polyamides, polyesters and so on. Elastomeric materials which can be used in the handle include silicone rubbers. Mixtures of polymeric and elastomeric materials can be used.

### II. Methods of Use

Although not restricted to this purpose, the devices described herein are particularly adapted for measuring the spacing and angulation of a void in the spine, for instance as is needed in a corpectomy procedure. The corpectomy can be a partial corpectomy, a complete single-level corpectomy, or a multi-level corpectomy. In a partial corpectomy, only a part of the vertebral body is removed. In a single-level corpectomy, the surgeon typically removes a vertebral body and the adjacent discs. A multi-level corpectomy includes the removal of all or part of two or more adjacent vertebral bodies. An implant or cage is typically inserted into the void created by the removal of all or part of the one or more vertebral bodies.

Corpectomy cages are known in the art. Corpectomy cages typically are used with end caps that are attached to each end of the substantially cylindrical body. The end plates are relatively flat structures with a central opening. Spinal implants are typically made of a biologically inert material, for example, any metal customarily used for surgical devices such as titanium or stainless steel. The end plates can have different sizes and angulations. By providing a precise measurement of the distance and angulation of the void, the orthopedic surgeon can select a corpectomy cage or implant that accommodates the size and shape of the void. The cage or implant fits inside the space and maintains or restores the height of the spine or space between adjacent vertebral bodies.

In a preferred embodiment the device is used to measure the distance and angulation of a space or void in the spine. Typically the surgeon inserts the distal end of the device, preferably in a closed position. The device is then extended such that the contacting surfaces are flush with the edges of the space to be measured. This can be, for instance, the space between adjacent vertebral bodies for inserting an intervertebral cage or implant. Each of the contacting surfaces is contacted with a vertebral body defining the boundaries of the space in the patient's spine. In the open position, the gauges are preferably positioned such that the surgeon, with a single measurement, can accurately determine the distance and the angulation of the space to be measured. The surgeon can then select an implant based upon the distance and angle measured. The surgeon then removes the device and inserts into the space a cage or implant having a size and shape that correlates with the measured angle and distance. In preferred embodiments the patient is a human patient.

### III. Exemplary Dynamic Corpectomy Caliper

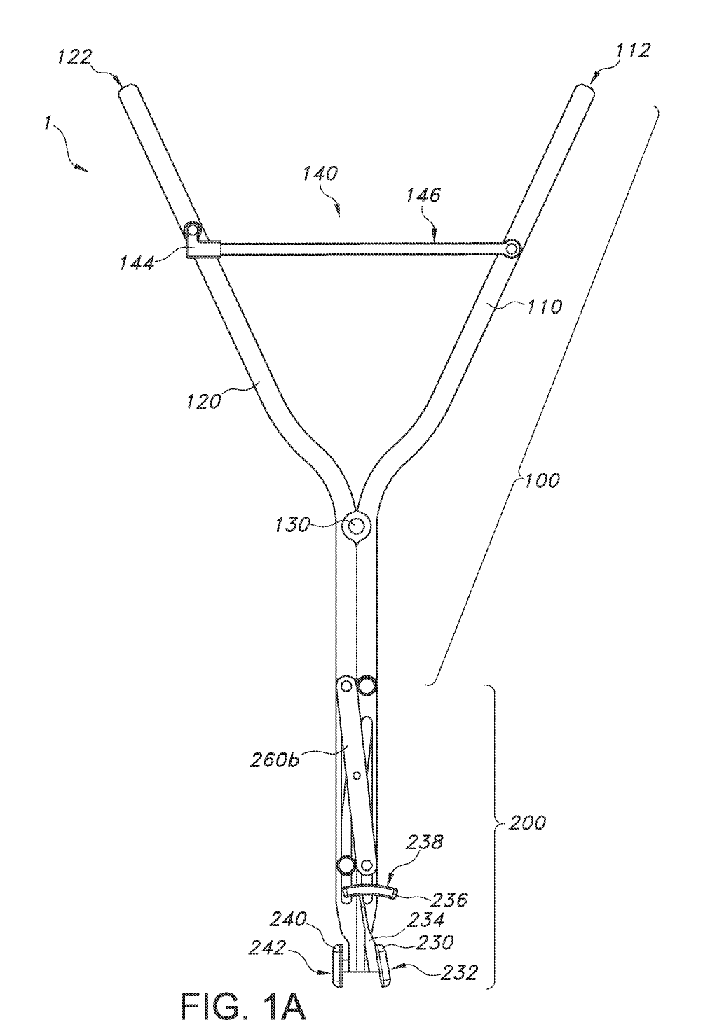

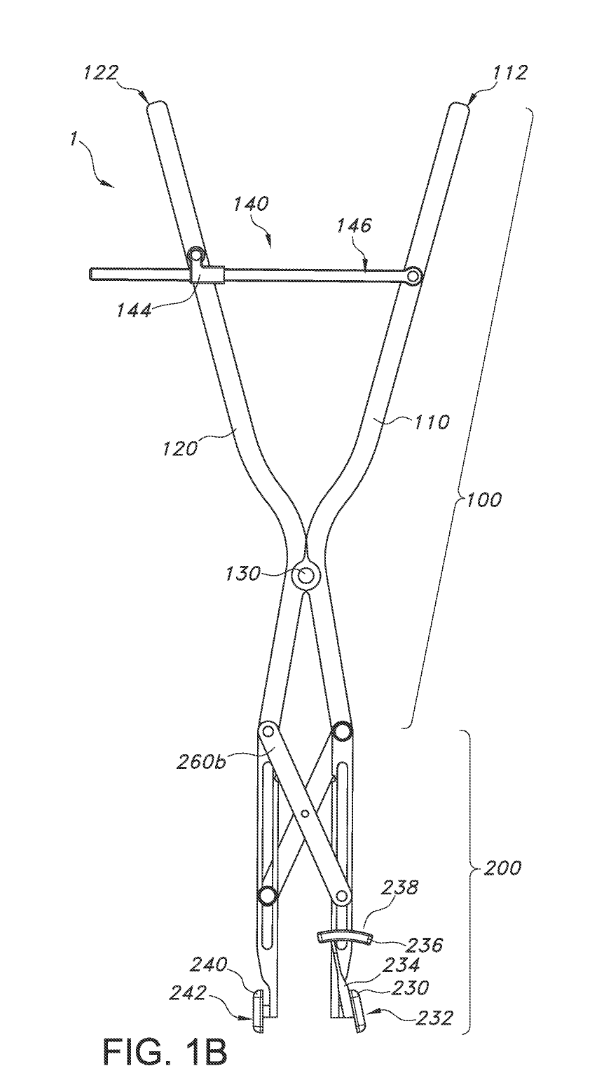

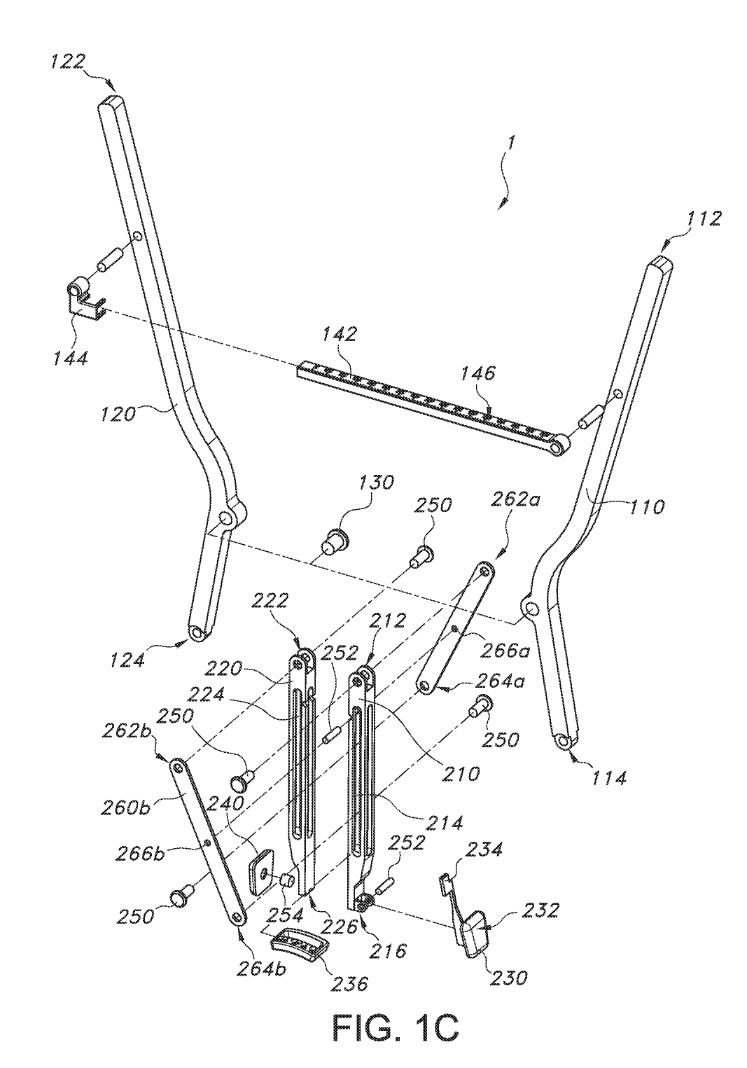

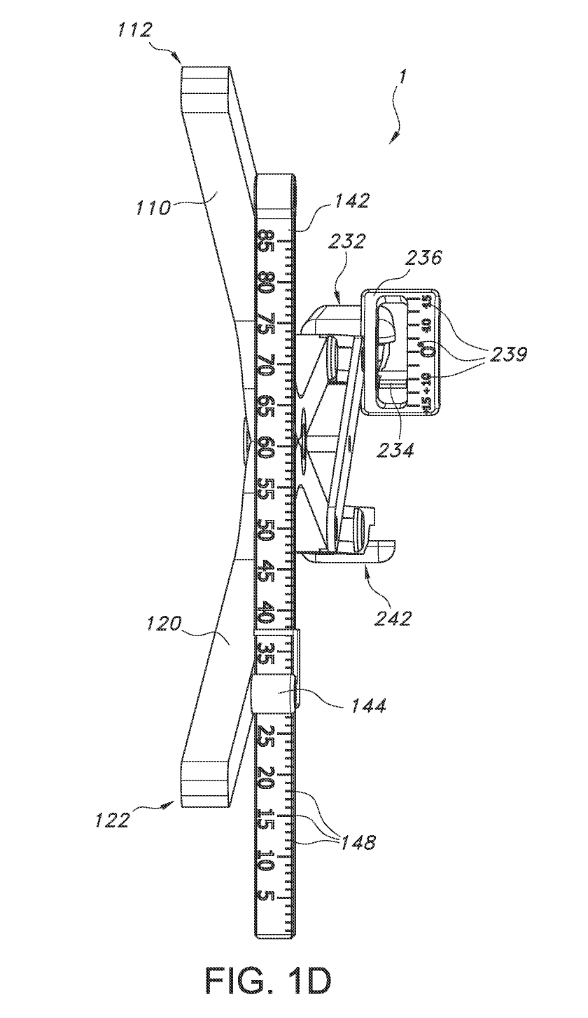

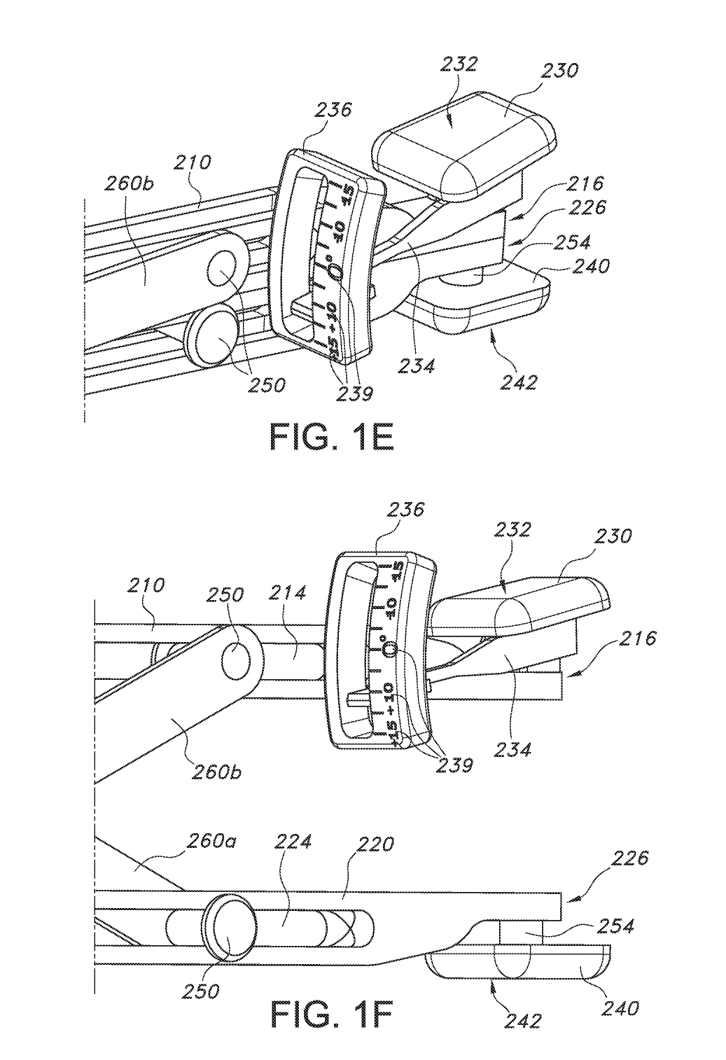

FIGS. 1A-1F depict an exemplary dynamic caliper device that can be used in a vertebral corpectomy. The caliper 1 has a caliper portion 100 and a contacting portion 200. The caliper portion 100 controls and measures the distance between a first contacting surface 232 and a second contacting surface 242.

In this embodiment, the caliper portion 100 has a first arm 110 and a second arm 120 operably engaged by a pivot pin 130. The first arm 110 has a handle end 112 and an engaging end 114. The second arm 120 has a handle end 122 and an engaging end 124. Each of the engaging ends 114 and 124 engages the contacting portion 200 in a manner that provides for control of the distance between the contacting surfaces 232 and 242. The caliper portion 100 contains a measurement portion 140 configured to determine or measure the distance between the contacting surfaces. For example as shown in device 1 the measurement portion 140 includes an elongated member 142 fixedly engaged with the first arm 110 and an indicator 144 operably engaged with the second arm 120. The indicator 144 is also slidably engaged with the elongated member 142. The elongated member 142 has a measurement surface 146 with a graduated scale 148 such that the position of the indicator 144 along the graduated scale 148 correlates with the spacing between the first contacting surface 232 and the second contacting surface 242.

The contacting portion 200 contains a first extension 210 pivotally engaged at a first end 212 with the engaging end 114 of the first arm 110 of the caliper portion 100, such as via a pin 250. The contacting portion contains a second extension 220 pivotally engaged at a first end 222 with the engaging end 124 of the second arm 120 of the caliper portion 100 via a pin 250.

The first extension 210 and the second extension 220 of the contacting portion 200 are maintained in a substantially parallel orientation by a pair of supports 260a and 260b. Each support contains two ends, one of which is a moveable end is connected to the engaging end of the caliper portion and the first end of the first end of the contacting portion, the second of which is engaged with a slot in the contacting portions. A support 260a is pivotally engaged at a fixed end 262a with both the first end 212 of the first extension 210 of the contacting portion 200 and the engaging end 114 of the first arm 110 of the caliper portion 100. Another support 260b is pivotally engaged at a fixed end 262b with both the first end 222 of the second extension 220 of the contacting portion 200 and the engaging end 124 of the second arm 120 of the caliper portion 100. The support 260a is slidably engaged at a movable end 264a with a slot 224 in the second extension 220 of the contacting portion 200 via a pin 250. The support 260b is slidably engaged at a movable end 264b with a slot 214 in the first extension 210 of the contacting portion 200 via a pin 250. The supports 260a and 260b are pivotally engaged in a scissor-like manner at a point (266a and 266b) on each that is intermediary to the fixed ends (262a and 262b) and the movable ends (262a and 264b) via a small pin 252.

The contacting portion 200 has a first plate 230 at the second end 216 of the first extension 210 and a second plate 240 at the second end 226 of the second extension 220. In this embodiment, the first plate 230 is hingedly engaged with the first extension 210, such as via a small pin 252. In this embodiment, the second plate 240 is fixedly engaged with the second extension 220 via a securing post 254. The first plate 230 has an indicator needle 234 slidably engaged with an indicator panel 236 that is fixed to the first extension 210. The indicator panel has a first face 238 containing a graduated scale 239 such that the position of the indicator needle 234 relative to the graduated scale 239 is indicative of the angle between the first contacting surface 232 and the second contacting surface 242.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

I claim:

1. A device comprising:
a distal end comprising a contacting portion comprising a first contacting surface and a second contacting surface;
a proximal end comprising a scissor-like caliper portion; and
one or more scales indicating the distance and the angle between the first contacting surface and the second contacting surface.

2. The device of claim 1, wherein the contacting portion comprises a first extension comprising the first contacting surface and a second extension comprising the second contacting surface.

3. The device of claim 2, wherein the first extension, the second extension, or both hingedly engage a plate comprising the first contacting surface or the second contacting surface.

4. The device of claim 2, wherein the first extension and the second extension of the contacting portion are configured such that they are maintained in a substantially parallel orientation when the device is in both an open and a closed position.

5. The device of claim 4, wherein the first extension of the contacting portion and the second extension of the contacting portion are engaged by one or more supports maintaining the relative orientation of the first arm to the second arm.

6. The device of claim 3, wherein the first extension hingedly engages a plate comprising the first contacting surface or the second extension hingedly engages a plate comprising the second contacting surface.

7. The device of claim 1, wherein the contacting portion comprises a mechanical gauge configured to measure the angle between the first contacting surface and the second contacting surface.

8. The device of claim 1, wherein the scissor-like caliper portion comprises a first arm pivotally engaged with a second arm, and
wherein the angle between the first arm and the second arm correlates with the distance between the first contacting surface and the second contacting surface.

9. The device of claim 8, wherein the caliper portion comprises a mechanical gauge configured to measure the distance between the first contacting surface and the second contacting surface.

10. The device of claim 1 made in whole or in part from a nondegradable, biologically inert material, such as stainless steel.

11. A method of using a device for measuring a space in a patient's spine, wherein the device comprises:
a distal end comprising a contacting portion comprising a first contacting surface and a second contacting surface;
a proximal end comprising a scissor-like caliper portion; and
one or more scales indicating the distance and the angle between the first contacting surface and the second contacting surface,
the method comprising:
(i) inserting the distal end of the device into the space,
(ii) contacting the first contacting surface with a first body and contacting the second contacting surface with a second body, and
(iii) measuring the distance and the angle between the first contacting surface and the second contacting surface from the one or more gauges.

12. The method of claim 11, wherein the first body and the second body define the boundaries of the space in the patient's spine.

13. The method of claim 11, wherein the first body and the second body are vertebra in the spine.

14. The method of claim 11, wherein the patient is a human patient.

15. The method of claim 11, further comprising:
(iv) selecting an implant for insertion into the space having a suitable size and shape to fit inside the space.

16. The method of claim 11, further comprising:
(iv) removing the device; and
(v) inserting into the space an implant with a suitable size and shape to fit inside the space.

17. The method of claim 16, wherein the measurement step (iii) and the insertion step (v) are not repeated.

18. The method of claim 11 performed as part of a corpectomy procedure.
